(19) United States
(12) Patent Application Publication
(10) Pub. No.: US 2007/0032647 A1
(43) Pub. Date: Feb. 8, 2007

(54) Novel process for preparation of 10-oxo-10, 11-dihydro-5h-dibenz [b,f] azepine-5-carbox- amide (oxcarbazepine) via intermediate, 10-methoxy-5h-debenz[b,f] azepine-5-carbonyl- chloride

(75) Inventors: Chandrashekar Parenky, Maharashtra (IN); Rohit Chaturvedi, Maharashtra (IN)

Correspondence Address: WELSH & KATZ, LTD, 120 S RIVERSIDE PLAZA, 22ND FLOOR, CHICAGO, IL, 60606, US

(73) Assignee: Amoli Organics Ltd., Mumbai (IN)

(21) Appl. No.: 10/576,546

(22) PCT Filed: Oct. 15, 2004
(86) PCT No.: PCT/IN04/00322

§ 371 Date: Apr. 20, 2006

(51) Int. Cl. C07D 223/18

(52) U.S. Cl. 540/589

(57) ABSTRACT

A process for preparation of 10-oxo-10,11-dihydro-5H-dibenz[b,f]azepine-5-carboxamide (oxcarbazepine) via intermediate 10-methoxy-5H-dibenz[b,f]azepine-5-carbonyl chloride, comprising the steps: a) Preparation of an intermediate 10-methoxy-5H-dibenz[b,f]azepine-5 carbonyl, chloride from 10-methoxyiminostillbene using bis (trichloromethyl) carbonate (BTC) with organic base such as aliphatic or aromatic tertiary amines in organic solvent; b) Conversion of the intermediate to 10-methoxy-5H-dibenz[b,f]azepine-5-carboxamide using ammonia in organic solvent; c) Formation of oxcarbazepine from step (b) using Bronsted acid in an organic solvent at a temperature between 25° C.-80° C., preferably at 50° C. to 70° C.; and d) Isolation of oxcarbazepine.

### FIELD OF THE INVENTION

The present invention relates to an improved process for preparation of 10-methoxy-5H-dibenz[b,f]azepine-5-carbonyl chloride from 10-methoxy-5H-dibenz[b,f]azepine (10-methoxy iminostilbene) without the use of phosgene and its further conversion to 10-oxo-10,11-dihydro-5H-dibenz[b,f]azepine-5-carboxamide (oxcarbazepine) without the use of strong mineral acids.

### BACKGROUND AND PRIOR ART

Oxcarbazepine is an anticonvulsant drug used as an anti-epileptical agent in treatment of AIDS-related neural disorders and for treatment of Parkinson's disease

Several processes for preparing Oxcarbazepine have been reported.

U.S. Pat. No. 3,462,775 describes the preparation of oxcarbazepine from 10-methoxy iminostilbene by phosgenation in toluene, followed by amidation (ethanol and ammonia) and hydrolysis in acidic medium to get e desired product (Scheme 1). The phosgenation is carried out at relatively high temperatures of around 95° C. and the hydrochloric acid produced leads to the formation of undesirable impurities. The process uses phosgene gas, which is toxic and hazardous requiring extreme precaution making this process commercially unattractive.

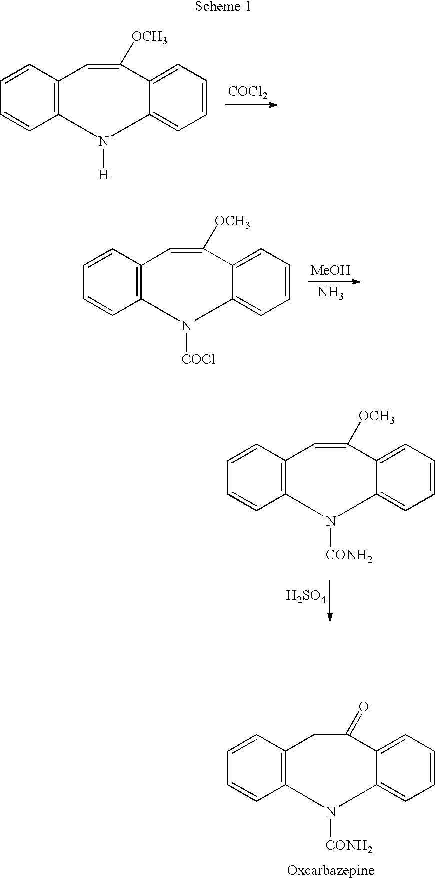

Canadian Patent 112 241 describes an alternate preparation of oxcarbazepine from the catalysed re-arrangement of 10,11-epoxycarbamazepine, prepared from carbamazepine by reaction with m-chloroperbenzoic acid (CPBA) (Scheme-2). Starting with Carbamazepine, which is an expensive raw material, the conversion to its epoxide is poor in quality and yield.

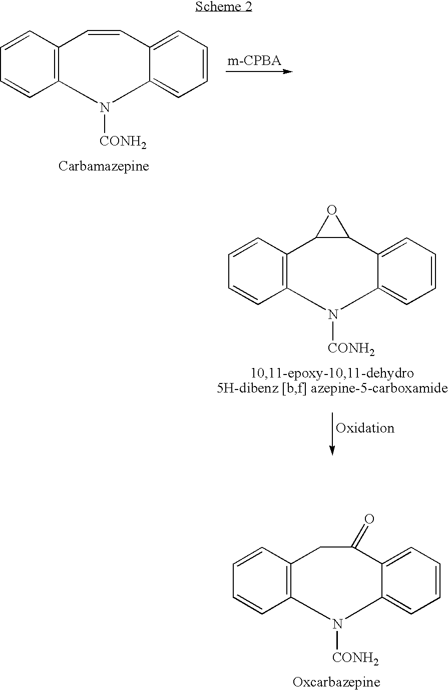

EP Patent Application 028028, discloses a process involving nitration of 5-cyanoiminostilbene followed by reduction and hydrolysis (Scheme-3). However, the drawback of the process is in the preparation of the 5-cyanoiminostilbene itself, which can be made from iminostilbene and cyanogen chloride. The latter is also toxic, hazardous and difficult to handle.

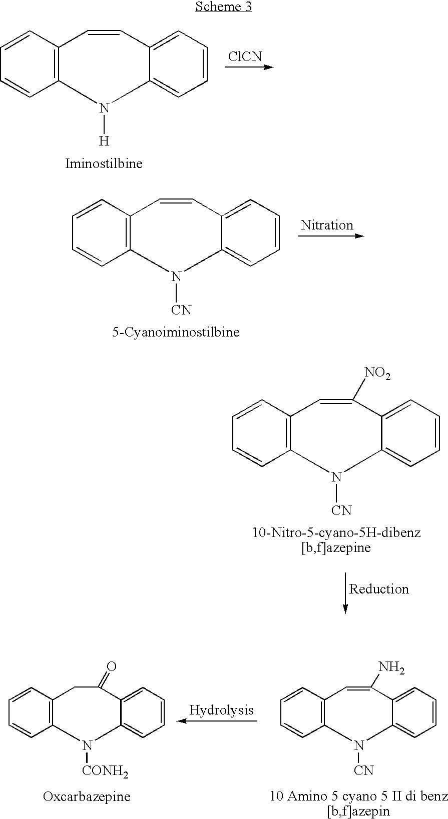

Swiss Patent No. 642 950 suggests hydrolysis of the 10-chloro-5H-dibenz[b,f]azepin-5-carboxamide using concentrated sulphuric acid to form the oxcarbazepine. However the yields are poor.

Further it may be noted that in all the processes disclosed in the prior art discussed above (Scheme 1 and Scheme 3) and U.S. Pat. No. 5,808,058, EP Application 1 302 464 A1 and PCT Publication WO 01156992A2, the conversion of 10-methoxy-5H-dibenz[b,f]azepine-5-carboxamide to 10-oxo-10,11-dihydro-5H-dibenz[b,f]azepine-5-carboxamide (oxcarbazepine) is effected using strong mineral acids or mixture of mineral acids and acetic acid in aqueous medium. This leads to degradation of oxcarbazepine

Methods described in the prior art have severe limitations in terms of poor quality and yields and also in some cases with the use of hazardous materials such as phosgene that need extreme care during usage making them commercially unattractive. Moreover the HCl formed during the course of the reaction and the relatively higher temperatures used leads to formation of undesired impurities.

There is a long standing need in the industry to provide cost effective, safe and easy operative processes for the production of 10-methoxy-5H-dibenz[b,f]azepine-5-carbonyl chloride from 10-methoxy-5H-dibenz[b,f]azepine (10-methoxy iminostilbene) without the use of phosgene and its further conversion to 10-oxo-10,11-dihydro-5H-dibenz[b,f]azepine-5-carboxamide (oxcarbazepine) without the use of mineral acids.

### SUMMARY OF THE INVENTION

The main object of the invention is to provide a cost effective, safe and high yielding process for the production of 10-methoxy-5H-dibenz[b,f]azepine-5-carbonyl chloride, from 10-methoxy-5H-dibenz[b,f]azepine (10-methoxy iminostilbene) without the use of phosgene gas as is practiced in the prior art an important intermediate for the synthesis of 10-oxo-10,11-dihydro-5H-dibenz[b,f]azepine-5-carboxamide (oxcarbazepine). It is further an object of the invention to provide a process for the conversion of the intermediate 10-methoxy-5H-dibenz[b,f]azepine-5-carboxamide to 10-oxo-10,11-dihydro-5H-dibenz[b,f]azepine-5-carboxamide (oxcarbazepine) without the use of mineral acids.

Another object of the invention is to develop a process that can be carried out at relatively lower temperatures to avoid the formation of any undesirable impurities.

Yet another object of the invention is to provide a cost effective process using easily available raw materials.

Yet another object of the invention is to provide a process for the conversion of the intermediate 10-methoxy-5H-dibenz[b,f]azepine-5-carboxamide to 10-oxo-10,11-dihydro-5H-dibenz[b,f]azepine-5-carboxamide (oxcarbazepine) using mild reagents such as methane sulphonic acid, para toluene sulphonic acid, Lewis acids, cationic resins, etc.

### DETAILED DESCRIPTION OF INVENTION

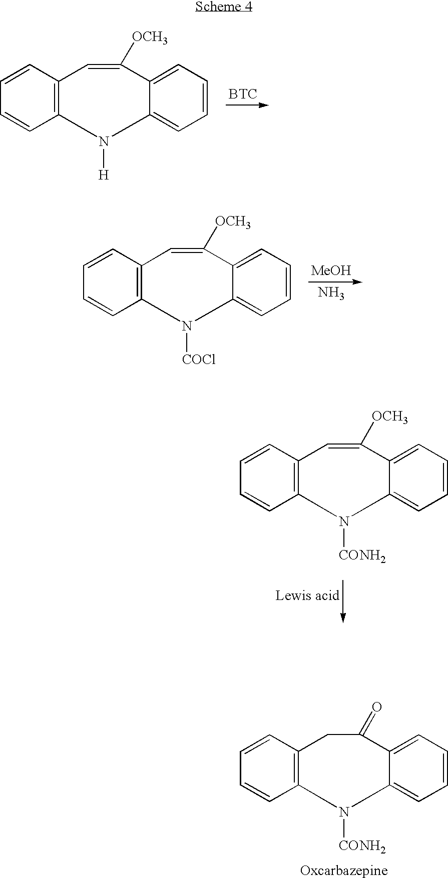

Thus in accordance of this invention the reaction (scheme 4) comprises steps
Preparation of intermediate 10-Methoxy-5H-dibenz[b,f]azepine-5-carbonyl chloride from 10-methoxyiminostilbine using bis-(trichloromethyl) carbonate (BTC) or triphosgene and an appropriate base in the presence of an organic solvent.
Conversion of 10-Methoxy-5H-dibenz[b,f]azepine-5-carbonyl chloride to 10-Methoxy-5H-dibenz[b,f]azepine-5-carboxamide using ammonia in a suitable organic solvent
Formation of oxcarbazepine from 10-Methoxy-5H-dibenz[b,f]azepine-5-carboxamide using Lewis acids in appropriate organic solvent.

10-Methoxyiminostilbene is dissolved in a solvent and cooled below 10° C. and bis-(trichloromethyl) carbonate (BTC) is added. An organic base is slowly added to the above solution over a period ranging from 3-24 hours maintaining the temperature below 10° C. till the reaction goes to completion. Optionally on completion of the base addition the reaction mixture is allowed to warm up to around room temperature and maintained at this temperature till the completion of the reaction as monitored by TLC/HPLC. On completion of the reaction, the reaction mixture is quenched in water and the layers are allowed to separate. The organic layer is separated and distilled to obtain crude 10-methoxy-5H-dibenz[b,f]azepine-5-carbonyl chloride which is purified using an organic solvent.

In the subsequent step 10-Methoxy-5H-dibenz(b,f)azepine-5-carbonyl chloride is refluxed in an aprotic solvent and ammonia gas is purged till the reaction goes to completion. The solvent is distilled and water is added, cooled to room temperature to isolate the 10-Methoxy-5H-dibenz[b,f]azepine-5-carboxamide.

10-Methoxy-5H-dibenz[b,f]azepine-5-carboxamide is stirred in an organic solvent in the presence of a Lewis acid at temperature upto 80° C. depending on the solvent used. On completion of the reaction the reaction mixture is cooled to room temperature and the crude oxcarbazepine is separated and purified.

The solvent used in the carbonyl chloride preparation step may be selected from chlorinated aliphatic hydrocarbons such as methylene dichloride, chloroform, ethylene dichloride, 1,1,1,-trichloroethane, trichloroethylene etc. or aromatic hydrocarbon solvent such as toluene, xylene, chlorobenzene, etc. or aprotic solvents including Dimethyl formamide, dimethyl acetamide, N-methyl pyrrolidine and acetonitrile. The organic base used in this step is selected from aliphatic/aromatic tertiary amines such as triethyl amine/diethyl aniline, pyridine, picoline etc.

In an embodiment of the process initial addition of the base may be followed by the addition of BTC.

The time of the addition of base ranges from 3-8 hrs, the temperature at which the base is added may range upto 30° C. preferably below 10° C. and most preferably from 0° to +5° C. The reaction period may vary from about 3 hours to about 10 hours. The molar ratio of 10-methoxy iminostilbene to BTC is 1:0.34-0.5. The molar ratio of 10-methoxy iminostilbene verses the base is 1:1-1.5.

The solvents preferred in the amidation reaction are selected from solvents like acetone, methyl cellosolve, methanol, ethanol, isopropyl alcohol, dimethyl formamide, dimethlacetamide, N-methyl pyrrolidone or aromatic solvents like toluene, xylene etc.

The solvent used in the final oxo preparation step ay be selected from chlorinated aliphatic hydrocarbons such as methylene dichloride, chloroform, ethylene dichloride, 1,1,1,-trichloroethane, trichloroethylene etc or aromatic hydrocarbon solvent such as toluene, xylene, chlorobenzene, etc. or aprotic solvents including dimethyl formamide, dimethyl acetamide, N-methyl pyrrolidine and acetonitrile.

The Lewis acids used in this are selected from cationic resins, para-toluene sulfonic acid, aluminium chloride, etc.

The temperature at which the reaction may be carried out may vary from 25 to 80° C., preferably between 50 to 70° C.

The invention is now illustrated with a few non-limiting examples.

### EXAMPLE 1

### Step 1. Preparation of 10-Methoxy-5H-dibenz[b,f]azepine-5-carbonyl chloride

100 gms of 10 Methoxy iminostilbene is dissolved in 300 ml chloroform & cooled to 0° C. Bis (trichloro methyl) carbonate (BTC) 65 gms is added. 67 gms of triethyl amine (TEA) in 100 ml chloroform is added slowly over a period of 6 hour & maintaining the temperature 0-5° C. Temperature is then increased to 25-30° C. 1 & maintained for 8 hour. The reaction mixture is poured into 300 ml water & layers are separated. Chloroform is evaporated 10-Methoxy-5H-dibenz[b,f]azepine-5-carbonyl chloride is isolated in methanol. Yield obtained is 110 gms (86%) of theoretical.

### Step 2. Preparation of 10-Methoxy-5H-dibenz[b,f]azepine-5-carboxamide from 10-Methoxy-5H-dibenz[b,f]azepine-5-carbonyl chloride

100 gm of 10-Methoxy-5H-dibenz[b,f]azepine-5-carbonyl chloride is refluxed in 500 ml methanol. Dry ammonia is passed into the boiling solution for 2 hours. The methanol is distilled water added and the reaction mixture is cooled to 25-30° C. and filtered. Yield of 10-Methoxy-5H-dibenz[b,f]azepine-5-carboxamide is 82 g.

### Step 3 Preparation of oxcarbazepine from 10-Methoxy-5H-dibenz(b,f)azepine 5-carboxamide

85 gm of 10-Methoxy-5H-dibenz[b,f]azepine-5-carboxamide is dissolved in 425 ml of ethylene dichloride. To this 800 ml of 2N o-toluene sulfonic acid is added and heated to 75-80° C. & maintained for bout 3 hours. It is then cooled to 20° C. & maintained for about 1 hour. The product oxcarbazepine is separated by filtration. This is then purified in acetone-water to yield 55 gms of pure oxcarbazepine.

### EXAMPLE 2

### Step 1. Preparation of 10-Methoxy-5H-dibenz[b,f]azepine-5-carbonyl chloride

100 gms of 10-Methoxy iminostilbine is dissolved in 300 ml chloroform & cooled to 0° C. 65 gms of Bis (trichloro methyl) carbonate (BTC) is added to the solution followed by the addition of 54 gms of Dimethyl aniline in 100 ml chloroform over a period of 4 hours maintaining the temperature 0-5° C. The temperature is then maintained 0-10° C. & maintained for 2 hours. The reaction mixture is poured into 300 ml water & layers are separated. Chloroform is evaporated & product is isolated in methanol. Yield obtained is 104 gms (82% of theoretical).

### EXAMPLE 3

### Step 1. Preparation of 10-Methoxy-5H-dibenz[b,f]azepine-5-carbonyl chloride

100 gms of 10-Methoxy iminostilbene is dissolved in 300 ml chloroform & cooled to 0° C. and 45 gms Bis (trichloro methyl) carbonate (BTC) is added followed by he addition of 45 gms of TEA in 100 ml chloroform over a period of 8 hours maintaining the temperature at 0-5° C. The temperature is then increased to 25-30° C. & maintained for 2 hours. The reaction mixture is poured into 300 ml water layers are separated. Chloroform is evaporated & product is isolated in methanol. Yield obtained is 100 gms (80% of theoretical):

The present invention obviates the use of phosgene gas in the preparation of 10-methoxy-5H-dibenz[b,f]azepine-5-carbonyl chloride from 10-methoxy-5H-dibenz[b,f]azepine (10-methoxy iminostilbene). Further the invention provide a process for the conversion of the intermediate 10-methoxy-5H-dibenz[b,f]azepine-5-carboxamide to 10-oxo-10,11-dihydro-5H-dibenz[b,f]azepine-5-carboxamide (oxcarbazepine) without the use harsh conditions and strong mineral acids thereby obtaining high quality oxcarbazepine in a cost effective manner from easily available raw materials.

1. A novel process for preparation of 10-oxo-10,11-dihydro-5H-dibenz[b,f] azepine-5-carboxamide (oxcarbazepine) via intermediate 10-methoxy-5H-dibenz[b,f]azepine-5-carbonyl chloride, comprising the following steps:
a) Preparation of an intermediate 10-methoxy-5H-dibenz[b,f]azepine-5-carbonyl chloride, from 10-methoxyiminostillbene using bis (trichloromethyl) carbonate (BTC) triphosgene with organic base/organic solvent
b) Conversion of above intermediate to 10-methoxy-5H-dibenz[b,f]azepine-5-carboxamide using ammonia and with suitable solvent.
c) Formation of oxcarbazepine from step (b) using Lewis acid in an appropriate organic solvent at a suitable temperature between 25-80° C. preferably at 50to 70° C.,
d) Isolation using organic solvent,

2. A novel process as claimed in claim 1, wherein at step (a) organic base is slowly added to the solution for a period of 3-24 hrs, maintaining a temperature at 10° C., after completion of reaction, mixture is allowed to rise to room temperature, followed by separation of organic layer, and distilled to get crude intermediate, purified using organic solvent.

3. A novel process as claimed in claim 1 & 2, wherein the ammonia gas is purged till the reaction completion, distilled the solvent, added water, followed by cooling at room temperature to isolate intermediate,

4. A novel process as claimed in the above claims, wherein the solvent selected is from chlorinated aliphatic hydrocarbons/aromatic hydrocarbons or aprotic solvent in the preparation of carbonyl chloride,

5. A novel process as claimed in claim 4, wherein chlorinated aliphatic solvents are such as methylene dichloride, chloroform, ethylene dichloride, 1,1,1,-trichloroethane, trichloroethylene etc.

6. A novel process as claimed in claim 4, wherein the solvent aromatic hydrocarbons are selected from toluene, xylene, chlorobenzene, etc.

7. A novel process as claimed in claim 4, wherein the aprotic solvents are selected from Dimethyl formamide, Dimethyl acetamide, N-methyl pyrrolidine and acetonitrile.

8. A novel process as claimed in claim 1 & 2 wherein the organic base is selected from aliphatic/aromatic tertiary amines.

9. A novel process as claimed in above claims, wherein the molar ratio of 10-methoxy iminostilbene to BTC is 1:0.34-0.5, and the molar ratio with base is 1:1-1.5.

10. A novel process as claimed in claim 9, wherein the solvent selected from acetone, methyl cellulose, methanol, ethanol, isopropyl alcohol, dimethylforamamide etc.

11. A novel process as claimed in claim 1, wherein the Lewis acid is selected from p-toluene sulfonic acid, cationic resins etc.

12. A novel process for preparing 10-oxo-10,11-dihydro-5H-dibenz[b,f]azepine-5-carboxamide (oxcarbazepine) via intermediate 10-methoxy-5H-dibenz[b,f]azepine-5-carbonyl chloride substantially therein described with reference to foregoing examples.
